SEQUENCE LISTING (3 sequences)

SEQ ID NO: 1 - DNA, 9490 bp, unidentified
  source 1..9490: mol_type = other DNA
accggtggcccggccccaaccccatgcgtcaagggatcaaaccatggcgcgtgtgcacatcgccaccgtcggcgctgcccagtcaccgcgccgacggcgaaatatattccggggtcccacgtggaagcatccggaggaatacacggatacacctccgcctcggtccatcgcaactgcaatcttctgctccgtcctgtaggctgtagctgcagcctgcagggagcgagccataaaggagactacgtagcggccgcggagcgcggggacgacggagggagacagacgggcgcggcagggaccagggagccagcagggctaggcgacgctccttccagttctcctatccatgatttcattctgtgagttgagggtgctctgctcctttccgtcccgtcaatttctgggttccatttcggtcgcttcttgcggagatcaaatgataccgcgcagattcgttcttctctagtttgggaaactcattttccggaattttcatttttccaaagaaaattagtttattttccttggaaagataggacttcggagtttgttacttccctcctctctttccccatgattcttcatgctctgcgaggtcaactgacgtgtataagaggagcactacttcttgcgttctcacatgtagctttagatttttgttgcttcatcgctatgtgctttcaatagtatatagcatattataaacttatatatatatatatatatataatttctcgtatataacccgtgtatctcggcttcatatgacttttcaagtactatgactgtgttggtatctattgcagcttcatatatacgtaagattgctttgcttgcttagtaattatcacgcaccattcaccaatctatgcttatgatagtgcgtcaagtatgcgtaggggtggcacgatccaaatgtttcttatgacttttaataaattttattagtttaaaaataaataggaatagagcccgactcttctaatctgatttgatccataagttttatatactctaaaatttagagcccattaccaccctaagtatatgtatgtatgactattcctgctcaatgtaatccaagtatttttctccacacctatttttatgcttccaaaggctccacgctccaaggatgatagcactattgaaccctcaaaacccatagctgccactcttctctgaacccctcctctagggattgaaggcaaggtcgagtctcataaaaagcacacctatttctaagcttccaaagctccacgctccaaggatgacagcactatggaatccttttattttgcctttgggaatttttaaagaagctttcctccaccagtcaacaaagtagacatctcttctcttaggtaccacagcagtaaggccaatttgggataaggtagcatgccataaccgccttgcaaagacatagttagttaagatatgctgcacagtttcttcttatcacaaaggacacaattcttgggaaagtcaagccctcttttttgaagtctgtctgctgtccaacatttaaaaaaaatgtaatcccagtagtttggttgtgcgccttgtccggccctgtgtcactcgcctctctgacagagggaagggctggtgttagtgggacgtcaggcttgtaatgttaaaaccctactgtcctcttaatggaatactggcttctgttatattttttttaacatactagcttcggcctgctcgcgaaaaaaagactataactgctcaacttatgaactatgaaccacaggtaatgcctaattcactaggctgcctattaaggtgaatgcgagcttagtctatccttgatcgaaattggctcacacctactgactactgtatatggttttaagcaattccatggtcaagctaatgtgtttgttacaacttttaagctttaaccacttatggcataaaggctttttgcctagcatgactgtttgccctgttgatcatgttttttacactttgtcgtactcatatctgtggttttttgctcatatttgaactctttcctttttagttgacagccagaattcaagccattgcgttcaaaaaatatatctgtgattttttgttcatgtggttcaattgcaggagacattcacatgcttccatgtgattgaccttgtccctaatgttctgatcaagattgccatggttgaaaggctatagtttcttatggtccatgtccacgattcaagaataaaatattgtttggcccatccattgtgataatggtctggttgcaagaccagctggaaatgcacaagttccactggtacagtagagcccctactccatcagagttctggataccacttgctgcttggttcactgttggcctagtcggtctttggacattttttcatttcttttcagtgtggcggcgaaagatcagtttaagctggatgaaaataattgccagatcgaagaggaagaattttgaaagaaaccatgaggttcctactgctgaacatgtttggaacacagaatcattgattcgtgcaaaaggaatgatgtgctgtgtatgcttggaatctatctcacctgctcagccccttgggcagatgatgacatcagaaaatatggttcaccgctgcaatgtttgcggtgcagctgcgcacataatatgctcttcaaattctcagaaagactgcaaatgcatctcaatgtttgggttcaaacatgtggtccatcaatggactgtactctggacagacatagctgaccaatccgaagaaggccagtactgtagttactgtgaggagttatgcagtgagtcttttctgggaggtcctcccatatactgctgcatgtggtgccaacgattggtgcatgctgattgccagtctgccatggctactgaaaccggtgaaatttgcgaccttggtcccttcaagcgccttattctgtcgccactttttgtccgggccattagcaaacctggtagcatcttgagttctataactcatggagcaaatgagtttgcaaccactgtgcgggggcgtttaaaccgaactaaaaaggagaagcatcagaacagattttcatctgacagcaacgatgattcatcaagtgataccacgttgaactcaaaccagagggctggagaattaaaaacaattggcgacagtgctcaaaggagtcctgagaatgagcatgatagcagtgagagtgatggtagagaactcatatcggagtccagaaggctcggtaatgatgagaccggtgaagttaaacttaagtacgcattgtctgaattgcctgctgattccagaccccttctaatttttatcaacaagaaaagtggggctcagcgcggagatcttctcaagcacaagctacattttcttttgaatccggtgcaggtcatgatacttctcacacaaaactttactttaagtcgagtcacttctcagctaggcaacatggtatgctaccaatatattcaattttgtagaatatgctcattgcatcactagttgggtgcatgcaattacttaaaatcatccacttggtgctggatcagttcatattatgtgaggaaatcatcataatcaaccttctgtcatgtttctagtgatggcttcacttaaaaataaaatccatcttctctgtgctaatcagtagtaagtagtaaccgcatggtaaagagggagtggtaagcctagatttttttttggatgttaggtaattcagttagtataatttactgcatctacagtgtgtaaaaacttgaaccctttgaagaagcaacaccgtttgcctacatcttctgctgttggatcatgtgtaggagacttgttttccatcctactaaaacgtaggcacacagctgaacagaagctgtcaacgtgcttggaatttgaaggctagtgttctgtttttggttctgggttgggcctccaggccatcatctttctgcgggtagtggttcactagttgacgtgtttttggtccaacctgccttgtaagggcgtggagtatggggttgtggtctttctgcgggtaggggtctttcactagtgatgtgtttttggtccaacctgccttgtaaggccgtggagtatggggctgtggtctttctgcgggtagggctctttccctagtgatgtgtttttggtccaacctggcttgtaaggccatggggtacagtcatgtagatactgtcaaagggggtttgcctgcaatccttttgtcttatatccttctaatataattcagtaaaactcttgctggcctttcgggaaaaaatgttctgttttcatatgtattttagcacccaccttagtaaatgcttcagatatttatgatttctgtgtttctaatgtttcatatcattcatctaggtttttgaattaagttcttcacaagggccagaaacaggattatttttgttcagaaaggtaccacatttcagaatacttgtgtgtggtggtgatggtactgttggttgggttcttgacgcgatagataagcaaaattatgaatcacctccacccattgcaattcttccagctggcactggcaatgatctttcgagagttttatcatggggaggtggcctaggtgctgttgagaagcaaggtggcctttgcacaattttacatgacatagagcatgcagcagtcactatccttgatagatggaaggtgacagtagaagataagaaatcaaagaatgtgcttctagtaaagtacatgaacaactatctcggtaggtacacagaaacttcgtttatcggcgctccttgtcgaataaccttagcttactatggtgacaggtatcggttgtgatgcaaaagttgcccttgacattcataatctccgtgagggaaatcctgagaaattctacagtcaggtaatgtttgtactgtacccatcataagcaacatacagtagttggccattaattttaatgtttagcttcttcacaaatctatacttgtgaagtggtcacttttcacatttattgtttaagaaacaaaagtagtacagctgcctgttacttgaaatgtggtgttaatttgatgttatcagatgtaagttagggcttgtggatgcttgctgctcgaaatttcttctgaagatttaacttttatatctcctgtaaatatgtgaattagcttgtagaaccttttaagttttgaggtgcttgtaggcatattggagtattgggtgccattggttcatgcctaaactacatgtcattgttgagtatagtagtgcttaatggtgaaccaattgaactggacttgaattgacagtgttgactgaggcaatctgatcgcttcagtgtgataccttcagcttttggttgagtgggtgattatcataactagcccatcaagcaatgtgttgcttctacacgggctaataattttaaaaggacataattgttagaagtttgtggataaaatttgttcctagtgctattcagaatagcgtatctctgctctctatcgttcatattatgacgaaatattcatagatacttttatctttattcaattctggacttgtaccatctattctatatgcttttccatccccatacgtgctttcccctttgcgtagcacctccgtaccttagtttgagctgtgtttgacatggaaattgatatgatgtggtccgcatgatgcaatggtcgtatgtatcatgcatgtttaccttgagttagttctatatcaacaatgtagaagtgggtaacctagaagcatatgtaggaggactttgggatatatattgagatgtagaattattagcttacttttggttattttaataacattaaaattggggcaacttagatgaaaatttagcgaatagttcaaatcatctttcacaatgatataggtgagtaatttagatggaagatttagggggttcactttagggtattttcaaaatggcagatttggtaatttcgatataaatttaggaggttcctttcagaatgtcaaaagtggacaagtatctttgaaatacaaaatatgtattattgtcaatgatggtccgagtctactacaattaatggttggctattttcgttccatatgagaaaaatttctatgaggactggtttggcagcaatagggaaaaaacccatggaaaaaaaatccaccaatgagatttaaatggcacctcgatttccatggaggattttccccactaggatataatttcctgccaccctttggcaaccaaggatttagtccaaagctgttttagtcccaaaacatagccagcagcaactctatactactatattggatgagaactttatccatcatcagaaaatggttcatccatagctatgtgatgctataaataatacttcaaaaaatcaggaaaaatcaaacaaagtaaactgaagcatagtaaattggtttatccatcatcacatcacactttatccatcatcacaatagtttgtcaagcaaactgaagtagtaaaagacagtatacttgttccttcacaatagtttgtcaaacaaagcttttcatgagtcatgtgccagattgtgaagccaccatcaacaaaatacatctatttttcttactggagcaccaatagcaaactaaagtcactgtcaacagaagatgcgctcaaatttgttcctttagatagatatggtagaggcagcgacaggagatggaaagaggtactgcaaggaagcatagtgttaaaacaaacatgcaacaaatcaatcagaagtcggtatgcaaaaaattccaatcattggaacctacaagtggatgttaagcttatgacacctaacttaacatactttgactaccttatctccaaaatatagccactggcaactagctctaggaataatgaaaaacatagaagaaaaatatggagatatgcgttcacaggaagaagaaaatgtccatgtcaataggaaatgtccagacggacaccgacaagcccatatgccttcctccaaatccaatacattctgaccactagatgaactggcattgattggtgctagcgatcagttgtgaaattaatctacacacctccccaatcttttttcagattgcaaatcaatctacatgaattgcaaatctcatattttagtatcatatttcaatttttttcagattgcagtatcctccgatacttcctcacgtatattacagtatcacttgtactttttttatagatcgaatgttcgtgtaattaatatgtatgtagcaatgaaacaaatataggaagtatcaggcctgctatagatattcagaacactgccaatctacgatgtacagagcaatctgtaatggtacgactaggacacaaaggagcaggtccagatctgaaggtaggtaggtactgacctcgccggagcttgatcttcttcttgtgggtgatgccctgcacggctgcacggagatccggccttggagtcgtccggcggtctgcgtggccttgggtgatgccctgcatggagcttgatcttcttcctgttcgggtcggcggtctgcgtggccttggcgtcgtccggcgcgaagagcacgacgtcgatgccctgcacggagatcctgccgtcggtgtagatgtccgggtccaagaggtagcggatcttgtgcggcagaaaccagaatgaaacgtaccttgtgcggcagaaaccagaagcaaaccggaatcaaacgacggaggcgctcagggcgttggagcgacggaggtgctggtgcatagcgaagacgagacgaggcggcggagaatcgcctggcctcctaaggccagggttgtttccgacgggaaaatggaacgaatatctgcacgagattgggctgccaaatgacacacatgaataatccgaggacaattttctggtgggctttctcccaggagaaaaggccaggaaacccgctggattttgggctgccaaaccagtcctgatatgtcttttttcccccctctagcgcgtctcagtaagacattaaggactaacgcggaggctttaaagagaatctttagttattaaatattaagatgactcttatgtttctttgttgggcttcatcagttcttaaataaggtgctatatgcgagggaaggggccaagagcatcattgacagggcttttgtggacttaccatggcaagttcggttggaagtcgatggcaccgagatcgagataccagaggttgtgcgggagtgaattgtgactaattacatgtagaccaccgccattactacatggttagttgcactaaactactagttggctatggatccttcaggactcggaaggcgtgcttgtcgcgaacatcccgagctacatgggaggggttgacctgtggcaaaacgagggtgaggatcccgaagattttgacccgcagtcgatccatgacaagatgctcgaagtggtcagcattacaggagcatggcatctgggaacactccaggtaagtgaagtgacgatatttcagaatcctttgaaggatggagtgaatggtggggacttaaaatgaaaaagaactgctaaactgcatctaggttggactttctcgggcgcggaggatcgcgcaaggccagtcgatcaagatacagatgtttgcccctttccccgttcaagtcgacggtgagccttgggtccagcagccctgcacgctcaagatatctcatcacgggcaggtttactctgaccttgctcttcaccatgcaaatcacataatatataagggggtgttttgtttctacggactatttttagtccctctattttattccctaaattgccaaataaggaaattaaaactctatcggtaatttagggactagaaactaaacaccccccccccccctaagttgaattgctggttcattgtcagagtgtttgtggttgggttttcttactttgttttgttttcgttgtattcaggcttttatgttgaggagagcagttgaagagccactgggtcatgccgctgctatgatcaccgacgtccttgagcatgcagagtccagccatgtaatcactgcttcgcagaagaaagccctcctccgagaaatggctctacggctgtcatgaagcaagcgccgctcattcggacgtcgcagaagaacggtttcctgcctctgcctgcacatcggtggttagtacgaggatctgtgatctcaacatgagctcgatcatgttgttagcagaagatacaggcccggagggacgtgtccacgtgcacatagagtttttttttttggggtcatgttcacgttttggaaatagcgaggcaatgttgtgtgcagctatgtatggtgtgtagacctcgggtaagccagttccttccttctgtgcaaaactagttacgcctgcggcctgccattccgtgttaggttgactggatggatttttagggctgcgtcgctgccacttcggttggtgccccatatgtaaacgagatgggcttgaatgcttggttgtaacctttttttttaatgataggagctctgtccttt

SEQ ID NO: 2 - protein, 716 aa, unidentified
MVWLQDQLEMHKFHWYSRAPTPSEFWIPLAAWFTVGLVGLWTFFHFFSVWRRKISLSWMKIIARSKRKNFERNHEVPTAEHVWNTESLIRAKGMMCCVCLESISPAQPLGQMMTSENMVHRCNVCGAAAHIICSSNSQKDCKCISMFGFKHVVHQWTVLWTDIADQSEEGQYCSYCEELCSESFLGGPPIYCCMWCQRLVHADCQSAMATETGEICDLGPFKRLILSPLFVRAISKPGSILSSITHGANEFATTVRGRLNRTKKEKHQNRFSSDSNDDSSSDTTLNSNQRAGELKTIGDSAQRSPENEHDSSESDGRELISESRRLGNDETGEVKLKYALSELPADSRPLLIFINKKSGAQRGDLLKHKLHFLLNPVQVFELSSSQGPETGLFLFRKVPHFRILVCGGDGTVGWVLDAIDKQNYESPPPIAILPAGTGNDLSRVLSWGGGLGAVEKQGGLCTILHDIEHAAVTILDRWKVTVEDKKSKNVLLVKYMNNYLGIGCDAKVALDIHNLREGNPEKFYSQFLNKVLYAREGAKSIIDRAFVDLPWQVRLEVDGTEIEIPEDSEGVLVANIPSYMGGVDLWQNEGEDPEDFDPQSIHDKMLEVVSITGAWHLGTLQVGLSRARRIAQGQSIKIQMFAPFPVQVDGEPWVQQPCTLKISHHGQAFMLRRAVEEPLGHAAAMITDVLEHAESSHVITASQKKALLREMALRLS

SEQ ID NO: 3 - DNA, 1421 bp, unidentified
  source 1..1421: mol_type = other DNA
atgcaccagcacctccgtcgctccaacgccctgagcgcctccgtcgtttgattccggtttgcttctggtttctgccgcacaaggtacgtttcattctggtttctgccgcacaagatccgctacctcttggacccggacatctacaccgacggcaggatctccgtgcagggcatcgacgtcgtgctcttcgcgccggacgacgccaaggccacgcagaccgccgacccgaacaggaagaagatcaagctccatgcagggcatcacccaaggccacgcagaccgccggacgactccaaggccggatctccgtgcagccgtgcagggcatcacccacaagaagaagatcaagctccggcgaggtcagtacctacctaccttcagatctggacctgctcctttgtgtcctagtcgtaccattacagattgctctgtacatcgtagattggcagtgttctgaatatctatagcaggcctgatacttcctatatttgtttcattgctacatacatattaattacacgaacattcgatctataaaaaaagtacaagtgatactgtaatatacgtgaggaagtatcggaggatactgcaatctgaaaaaaattgaaatatgatactaaaatatgagatttgcaattcatgtagattgatttgcaatctgaaaaaagattggggaggtgtgtagattaatttcacaactgatcgctagcaccaatcaatgccagttcatctagtggtcagaatgtattggatttggaggaaggcatatgggcttgtcggtgtccgtctggacatttcctattgacatggacattttcttcttcctgtgaacgcatatctccatatttttcttctatgtttttcattattcctagagctagttgccagtggctatattttggagataaggtagtcaaagtatgttaagttaggtgtcataagcttaacatccacttgtaggttccaatgattggaattttttgcataccgacttctgattgatttgttgcatgtttgttttaacactatgcttccttgcagtacctctttccatctcctgtcgctgcctctaccatatctatctaaaggaacaaatttgagcgcatcttctgttgacagtgactttagtttgctattggtgctccagtaagaaaaatagatgtattttgttgatggtggcttcacaatctggcacatgactcatgaaaagctttgtttgacaaactattgtgaaggaacaagtatactgtcttttactacttcagtttgcttgacaaactattgtgatgatggataaagtgtgatgtgatgatggataaaccaatttactatgcttcagtttactttgtttgatttttcctgattttttgaagtattatttatagcatcacatagctatggatgaaccattttctgatgat

(19) United States
(12) Patent Application Publication
(10) Pub. No.: US 2026/0035711 A1
(43) Pub. Date: Feb. 5, 2026

(54) ZM00001D029550 GENE FOR REGULATING OIL CONTENT OF MAIZE KERNELS AND APPLICATION THEREOF

(71) Applicant: Institute of Food Crops, Yunnan Academy of Agricultural Sciences, Kunming (CN)

(72) Inventors: Xingming Fan, Kunming (CN); Mengfei Shi, Kunming (CN); Fuyan Jiang, Kunming (CN); Wenhua Yao, Kunming (CN)

(21) Appl. No.: 19/238,330

(22) Filed: Jun. 13, 2025

(30) Foreign Application Priority Data
Aug. 5, 2024 (CN) 202411068442.X

(51) Int. Cl. C12N 15/82

(52) U.S. Cl. CPC C12N 15/8247

(57) ABSTRACT

The disclosure relates to the field of agricultural biotechnology, and specifically relates to a Zm00001d029550 gene for regulating oil content of maize kernels and an application thereof. In the disclosure, temperate maize inbred line Ye107 with a lower kernel oil content is used as a common parent, which is crossed with 5 tropical and subtropical maize inbred lines that have higher kernel oil contents, to construct a maize multi-parent population with significantly different kernel oil contents. Genome-wide association study (GWAS) analysis and genetic linkage analysis are utilized to jointly locate SNP_75791466 which is on chromosome 1 and significantly associated with kernel oil content, and functional gene Zm00001d029550 that regulates kernel oil content is further identified. The results of the disclosure help to further study a regulatory mechanism of oil content of maize kernels, and also provide technical support for breeding of maize varieties with high quality and high oil content.

### CROSS-REFERENCE TO RELATED APPLICATION

This application claims priority of Chinese Patent Application No. 202411068442.X, filed on Aug. 5, 2024, the entire contents of which are incorporated herein by reference.

### REFERENCE TO A SEQUENCE LISTING SUBMITTED VIA EFS-WEB

The content of the xml file of the sequence listing named “HKIP-US-1-1353-26_sequence listing” which is 15,365 b in size was created on Jun. 6, 2025 and electronically submitted via EFS_Web herewith. These sequence listing is incorporated herein by reference in its entirety.

### TECHNICAL FIELD

The disclosure relates to the field of agricultural biotechnology, and particularly relates to a Zm00001d029550 gene for regulating oil content of maize kernels and an application thereof.

### BACKGROUND

Oil is one of the primary nutrients stored in maize kernels, and is also an important component of nutritional and economic value of the maize kernels. Enhancing oil content helps to optimize feed requirements by improving maize quality, and therefore the study on oil content which is a trait in maize is of great significance. Maize serves not only as a crucial oil crop in various regions worldwide but also as an important source of vegetable oil. Featuring relatively large seeds and embryos, and a higher content of polyunsaturated fatty acids, maize also serves as a source of quality oil. However, compared with other oil crops, the oil content in maize seeds is relatively low, so breeders analyze different maize genotypes and different oil accumulations to increase the percentage of oil content. In summary, enhancing oil content is a key objective in plant breeding and biotechnology-assisted improvement. Therefore, identifying functional genes closely associated with the oil content of maize kernels can provide technical support for molecular marker-assisted selection of maize with high quality and high oil content.

### SUMMARY

To solve the above problem, the disclosure provides a Zm00001d029550 gene for regulating oil content of maize kernels and an application thereof. The Zm00001d029550 gene discovered in the disclosure is a functional gene that regulates kernel oil content, and the gene can explain 1.4% of phenotypic variation of the kernel oil content.

To realize the above objective, the disclosure provides the following technical solutions.

The disclosure provides a Zm00001d029550 gene for regulating oil content of maize kernels, and an amino acid sequence of a protein encoded by the Zm00001d029550 gene is as shown in SEQ ID NO.2.

Preferably, a nucleotide sequence of the Zm00001d029550 gene is as shown in SEQ ID NO.1.

The disclosure provides an application of the Zm00001d029550 gene described in the above technical solution in regulating oil content of plant kernels or in plant breeding.

Preferably, the regulating the oil content of the plant kernels includes: improving an expression quantity of the Zm00001d029550 gene to improve the oil content of the plant kernels, or reducing the expression quantity of the Zm00001d029550 gene to reduce the oil content of the plant kernels.

Preferably, the plant breeding includes: cultivating plants with a high kernel oil content or cultivating plants with a low kernel oil content.

Preferably, the plants include gramincous plants.

Preferably, the gramincous plants include maize.

The disclosure provides a method for improving oil content of maize kernels, including:

over-expressing the Zm00001d029550 gene described in the above technical solution in maize.

The disclosure provides a method for reducing oil content of maize kernels, including:

reducing the expression quantity of the Zm00001d029550 gene described in the above technical solution in maize.

Preferably, the method for reducing includes: silence, knock or knock down.

### Advantageous Effects

The disclosure provides a Zm00001d029550 gene for regulating oil content of maize kernels and an application thereof. In the disclosure, temperate maize inbred line Ye107 with a lower kernel content is used as a common parent, which is crossed with 5 tropical and subtropical maize inbred lines with relatively high kernel oil content, to construct a maize multi-parent population with significantly different kernel oil contents. Genome-wide association study (GWAS) analysis and genetic linkage analysis are utilized to jointly locate SNP_75791466 which is on chromosome 1 and significantly associated with kernel oil content, and functional gene Zm00001d029550 that regulates the kernel oil content is further identified. The gene can explain 1.4% of phenotypic variation in kernel oil content. Haplotype analysis shows that, in 429 recombinant inbred lines (RILs), the Zm00001d029550 gene has 3 haplotypes (Hap1, Hap2 and Hap3). The kernel oil content of Hap3 is significantly higher than that of Hap1 and Hap2. Therefore, Hap3 of the Zm00001d029550 gene is the haplotype type that significantly increases kernel oil content. The results of the disclosure help to further study a regulatory mechanism of the oil content of maize kernels, and also provide technical support for breeding maize varieties with high quality and high oil content.

### BRIEF DESCRIPTION OF THE DRAWINGS

For describing the technical solutions of the embodiments in the disclosure or in the prior art clearer, the accompanying drawings required in the embodiments will be described briefly below.

FIG. 1a shows results of correlation between pop1 and total oil content (TOC) in three environments;

FIG. 1b shows results of correlation between pop2 and TOC in three environments;

FIG. 1c shows results of correlation between pop3 and TOC in three environments;

FIG. 1d shows results of correlation between pop4 and TOC in three environments;

FIG. 1e shows results of correlation between pop5 and TOC in three environments;

FIG. 2 shows phylogenetic trees of pop1, pop2, pop3, pop4 and pop5;

FIG. 3 shows principal component analysis (PCA) results of pop1, pop2, pop3, pop4 and pop5;

FIG. 4 shows genetic structures of pop1, pop2, pop3, pop4 and pop5;

FIG. 5 shows linkage disequilibrium (LD) decay results of 5 RIL populations;

FIG. 6a shows a Manhattan plot, presenting the results of oil content in 22YS;

FIG. 6b shows a quantile-quantile (QQ) plot, presenting the results of oil content in 22YS;

FIG. 6c shows a Manhattan plot, presenting the results of oil content in 22JH;

FIG. 6d shows a QQ plot, presenting the results of oil content in 22JH;

FIG. 6c shows a Manhattan plot, presenting the results of oil content in 21YS;

FIG. 6f shows a QQ plot, presenting the results of oil content in 21YS;

FIG. 6g shows a Manhattan plot, presenting the results of oil content by the best linear unbiased prediction (BLUP);

FIG. 6h shows a QQ plot, presenting the results of oil content by the BLUP;

FIG. 7 shows detection results of quantitative trait loci (QTL) on different chromosomes of maize in Jinghong in the subpopulation pop3;

FIG. 8 shows GWAS identification results;

FIG. 9 shows haplotype analysis results;

FIG. 10a shows haplotype analysis results in a column diagram;

FIG. 10b shows haplotype analysis results in a column diagram;

FIG. 10c shows haplotype analysis results in a box plot; and

FIG. 11 shows expression levels of a Zm00001d029550 gene in various tissues.

### DETAILED DESCRIPTION

The disclosure provides a Zm00001d029550 gene for regulating oil content of maize kernels, and an amino acid sequence of a protein encoded by the Zm00001d029550 gene is as shown in SEQ ID NO.2. In the disclosure, a nucleotide sequence of the Zm00001d029550 gene is preferably as shown in SEQ ID NO.1. The specific sequence is as follows:

SEQ ID NO. 1:
5′-accggtggcccggccccaaccccatgcgtcaagggatcaaaccatggcgcgtgtgcacatcgccaccgtcggcgctg
cccagtcaccgcgccgacggcgaaatatattccggggtcccacgtggaagcatccggaggaatacacggatacacctccgcctcggtccatc
gcaactgcaatcttctgctccgtcctgtaggctgtagctgcagcctgcagggagcgagccataaaggagactacgtagcggccgcggagcgc
ggggacgacggagggagacagacgggcgcggcagggaccagggagccagcagggctaggcgacgctccttccagttctcctatccatga
tttcattctgtgagttgagggtgctctgctcctttccgtcccgtcaatttctgggttccatttcggtcgcttcttgcggagatcaaatgataccgcgcag
attcgttcttctctagtttgggaaactcattttccggaattttcatttttccaaagaaaattagtttattttccttggaaagataggacttcggagtttgttac
ttccctcctctctttccccatgattcttcatgctctgcgaggtcaactgacgtgtataagaggagcactacttcttgcgttctcacatgtagctttagatt
tttgttgcttcatcgctatgtgctttcaatagtatatagcatattataaacttatatatatatatatatatataatttctcgtatataacccgtgtatctcggctt
catatgacttttcaagtactatgactgtgttggtatctattgcagcttcatatatacgtaagattgctttgcttgcttagtaattatcacgcaccattcacc
aatctatgcttatgatagtgcgtcaagtatgcgtaggggtggcacgatccaaatgtttcttatgacttttaataaattttattagtttaaaaataaatagg
aatagagcccgactcttctaatctgatttgatccataagttttatatactctaaaatttagagcccattaccaccctaagtatatgtatgtatgactattcc
tgctcaatgtaatccaagtatttttctccacacctatttttatgcttccaaaggctccacgctccaaggatgatagcactattgaaccctcaaaaccca
tagctgccactcttctctgaacccctcctctagggattgaaggcaaggtcgagtctcataaaaagcacacctatttctaagcttccaaagctccacg
ctccaaggatgacagcactatggaatccttttattttgcctttgggaatttttaaagaagctttcctccaccagtcaacaaagtagacatctcttctctta
ggtaccacagcagtaaggccaatttgggataaggtagcatgccataaccgccttgcaaagacatagttagttaagatatgctgcacagtttcttctt
atcacaaaggacacaattcttgggaaagtcaagccctcttttttgaagtctgtctgctgtccaacatttaaaaaaaatgtaatcccagtagtttggttg
tgcgccttgtccggccctgtgtcactcgcctctctgacagagggaagggctggtgttagtgggacgtcaggcttgtaatgttaaaaccctactgtc
ctcttaatggaatactggcttctgttatattttttttaacatactagcttcggcctgctcgcgaaaaaaagactataactgctcaacttatgaactatgaa
ccacaggtaatgcctaattcactaggctgcctattaaggtgaatgcgagcttagtctatccttgatcgaaattggctcacacctactgactactgtat
atggttttaagcaattccatggtcaagctaatgtgtttgttacaacttttaagctttaaccacttatggcataaaggctttttgcctagcatgactgtttgc
cctgttgatcatgttttttacactttgtcgtactcatatctgtggttttttgctcatatttgaactctttcctttttagttgacagccagaattcaagccattgc
gttcaaaaaatatatctgtgattttttgttcatgtggttcaattgcaggagacattcacatgcttccatgtgattgaccttgtccctaatgttctgatcaag
attgccatggttgaaaggctatagtttcttatggtccatgtccacgattcaagaataaaatattgtttggcccatccattgtgataatggtctggttgca
agaccagctggaaatgcacaagttccactggtacagtagagcccctactccatcagagttctggataccacttgctgcttggttcactgttggcct
agtcggtctttggacattttttcatttcttttcagtgtggcggcgaaagatcagtttaagctggatgaaaataattgccagatcgaagaggaagaattt
tgaaagaaaccatgaggttcctactgctgaacatgtttggaacacagaatcattgattcgtgcaaaaggaatgatgtgctgtgtatgcttggaatct
atctcacctgctcagccccttgggcagatgatgacatcagaaaatatggttcaccgctgcaatgtttgcggtgcagctgcgcacataatatgctctt
caaattctcagaaagactgcaaatgcatctcaatgtttgggttcaaacatgtggtccatcaatggactgtactctggacagacatagctgaccaatc
cgaagaaggccagtactgtagttactgtgaggagttatgcagtgagtcttttctgggaggtcctcccatatactgctgcatgtggtgccaacgattg
gtgcatgctgattgccagtctgccatggctactgaaaccggtgaaatttgcgaccttggtcccttcaagcgccttattctgtcgccactttttgtccg
ggccattagcaaacctggtagcatcttgagttctataactcatggagcaaatgagtttgcaaccactgtgcgggggcgtttaaaccgaactaaaa
aggagaagcatcagaacagattttcatctgacagcaacgatgattcatcaagtgataccacgttgaactcaaaccagagggctggagaattaaa
aacaattggcgacagtgctcaaaggagtcctgagaatgagcatgatagcagtgagagtgatggtagagaactcatatcggagtccagaaggct
cggtaatgatgagaccggtgaagttaaacttaagtacgcattgtctgaattgcctgctgattccagaccccttctaatttttatcaacaagaaaagtg
gggctcagcgcggagatcttctcaagcacaagctacattttttttgaatccggtgcaggtcatgatacttctcacacaaaactttactttaagtcga
gtcacttctcagctaggcaacatggtatgctaccaatatattcaattttgtagaatatgctcattgcatcactagttgggtgcatgcaattacttaaaat
catccacttggtgctggatcagttcatattatgtgaggaaatcatcataatcaaccttctgtcatgtttctagtgatggcttcacttaaaaataaaatcc
atcttctctgtgctaatcagtagtaagtagtaaccgcatggtaaagagggagtggtaagcctagatttttttttggatgttaggtaattcagttagtata
atttactgcatctacagtgtgtaaaaacttgaaccctttgaagaagcaacaccgtttgcctacatcttctgctgttggatcatgtgtaggagacttgttt
tccatcctactaaaacgtaggcacacagctgaacagaagctgtcaacgtgcttggaatttgaaggctagtgttctgtttttggttctgggttgggcct
ccaggccatcatctttctgcgggtagtggttcactagttgacgtgtttttggtccaacctgccttgtaagggcgtggagtatggggttgtggtctttct
gcgggtaggggtctttcactagtgatgtgtttttggtccaacctgccttgtaaggccgtggagtatggggctgtggtctttctgcgggtagggctctt
tccctagtgatgtgtttttggtccaacctggcttgtaaggccatggggtacagtcatgtagatactgtcaaagggggtttgcctgcaatccttttgtct
tatatccttctaatataattcagtaaaactcttgctggcctttcgggaaaaaatgttctgttttcatatgtattttagcacccaccttagtaaatgcttcaga
tatttatgatttctgtgtttctaatgtttcatatcattcatctaggtttttgaattaagttcttcacaagggccagaaacaggattatttttgttcagaaaggt
accacatttcagaatacttgtgtgtggtggtgatggtactgttggttgggttcttgacgcgatagataagcaaaattatgaatcacctccacccattg
caattcttccagctggcactggcaatgatctttcgagagttttatcatggggaggtggcctaggtgctgttgagaagcaaggggcctttgcacaat
tttacatgacatagagcatgcagcagtcactatccttgatagatggaaggtgacagtagaagataagaaatcaaagaatgtgcttctagtaaagta
catgaacaactatctcggtaggtacacagaaacttcgtttatcggcgctccttgtcgaataaccttagcttactatggtgacaggtatcggttgtgat
gcaaaagttgcccttgacattcataatctccgtgagggaaatcctgagaaattctacagtcaggtaatgtttgtactgtacccatcataagcaacata
cagtagttggccattaattttaatgtttagcttcttcacaaatctatacttgtgaagtggtcacttttcacatttattgtttaagaaacaaaagtagtacag
ctgcctgttacttgaaatgtggtgttaatttgatgttatcagatgtaagttagggcttgtggatgcttgctgctcgaaatttcttctgaagatttaactttta
tatctcctgtaaatatgtgaattagcttgtagaaccttttaagttttgaggtgcttgtaggcatattggagtattgggtgccattggttcatgcctaaact
acatgtcattgttgagtatagtagtgcttaatggtgaaccaattgaactggacttgaattgacagtgttgactgaggcaatctgatcgcttcagtgtg
ataccttcagcttttggttgagtgggtgattatcataactagcccatcaagcaatgtgttgcttctacacgggctaataattttaaaaggacataattgt
tagaagtttgtggataaaatttgttcctagtgctattcagaatagcgtatctctgctctctatcgttcatattatgacgaaatattcatagatacttttatctt
tattcaattctggacttgtaccatctattctatatgcttttccatccccatacgtgctttcccctttgcgtagcacctccgtaccttagtttgagctgtgttt
gacatggaaattgatatgatgtggtccgcatgatgcaatggtcgtatgtatcatgcatgtttaccttgagttagttctatatcaacaatgtagaagtgg
gtaacctagaagcatatgtaggaggactttgggatatatattgagatgtagaattattagcttacttttggttattttaataacattaaaattggggcaa
cttagatgaaaatttagcgaatagttcaaatcatctttcacaatgatataggtgagtaatttagatggaagatttagggggttcactttagggtattttc
aaaatggcagatttggtaatttcgatataaatttaggaggttcctttcagaatgtcaaaagtggacaagtatctttgaaatacaaaatatgtattattgt
caatgatggtccgagtctactacaattaatggttggctattttcgttccatatgagaaaaatttctatgaggactggtttggcagcaatagggaaaaa
acccatggaaaaaaaatccaccaatgagatttaaatggcacctcgatttccatggaggattttccccactaggatataatttcctgccaccctttgg
caaccaaggatttagtccaaagctgttttagtcccaaaacatagccagcagcaactctatactactatattggatgagaactttatccatcatcagaa
aatggttcatccatagctatgtgatgctataaataatacttcaaaaaatcaggaaaaatcaaacaaagtaaactgaagcatagtaaattggtttatcc
atcatcacatcacactttatccatcatcacaatagtttgtcaagcaaactgaagtagtaaaagacagtatacttgttccttcacaatagtttgtcaaaca
aagcttttcatgagtcatgtgccagattgtgaagccaccatcaacaaaatacatctatttttcttactggagcaccaatagcaaactaaagtcactgt
caacagaagatgcgctcaaatttgttcctttagatagatatggtagaggcagcgacaggagatggaaagaggtactgcaaggaagcatagtgtt
aaaacaaacatgcaacaaatcaatcagaagtcggtatgcaaaaaattccaatcattggaacctacaagtggatgttaagcttatgacacctaactt
aacatactttgactaccttatctccaaaatatagccactggcaactagctctaggaataatgaaaaacatagaagaaaaatatggagatatgcgttc
acaggaagaagaaaatgtccatgtcaataggaaatgtccagacggacaccgacaagcccatatgccttcctccaaatccaatacattctgacca
ctagatgaactggcattgattggtgctagcgatcagttgtgaaattaatctacacacctccccaatcttttttcagattgcaaatcaatctacatgaatt
gcaaatctcatattttagtatcatatttcaatttttttcagattgcagtatcctccgatacttcctcacgtatattacagtatcacttgtactttttttatagatc
gaatgttcgtgtaattaatatgtatgtagcaatgaaacaaatataggaagtatcaggcctgctatagatattcagaacactgccaatctacgatgtac
agagcaatctgtaatggtacgactaggacacaaaggagcaggtccagatctgaaggtaggtaggtactgacctcgccggagcttgatcttcttct
tgtgggtgatgccctgcacggctgcacggagatccggccttggagtcgtccggcggtctgcgtggccttgggtgatgccctgcatggagcttg
atcttcttcctgttcgggtcggcggtctgcgtggccttggcgtcgtccggcgcgaagagcacgacgtcgatgccctgcacggagatcctgccgt
cggtgtagatgtccgggtccaagaggtagcggatcttgtgcggcagaaaccagaatgaaacgtaccttgtgcggcagaaaccagaagcaaac
cggaatcaaacgacggaggcgctcagggcgttggagcgacggaggtgctggtgcatagcgaagacgagacgaggcggcggagaatcgc
ctggcctcctaaggccagggttgtttccgacgggaaaatggaacgaatatctgcacgagattgggctgccaaatgacacacatgaataatccga
ggacaattttctggtgggctttctcccaggagaaaaggccaggaaacccgctggattttgggctgccaaaccagtcctgatatgtcttttttccccc
ctctagcgcgtctcagtaagacattaaggactaacgcggaggctttaaagagaatctttagttattaaatattaagatgactcttatgtttctttgttgg
gcttcatcagttcttaaataaggtgctatatgcgagggaaggggccaagagcatcattgacagggcttttgtggacttaccatggcaagttcggtt
ggaagtcgatggcaccgagatcgagataccagaggttgtgcgggagtgaattgtgactaattacatgtagaccaccgccattactacatggtta
gttgcactaaactactagttggctatggatccttcaggactcggaaggcgtgcttgtcgcgaacatcccgagctacatgggaggggttgacctgt
ggcaaaacgagggtgaggatcccgaagattttgacccgcagtcgatccatgacaagatgctcgaagtggtcagcattacaggagcatggcatc
tgggaacactccaggtaagtgaagtgacgatatttcagaatcctttgaaggatggagtgaatggtggggacttaaaatgaaaaagaactgctaaa
ctgcatctaggttggactttctcgggcgcggaggatcgcgcaaggccagtcgatcaagatacagatgtttgcccctttccccgttcaagtcgacg
gtgagccttgggtccagcagccctgcacgctcaagatatctcatcacgggcaggtttactctgaccttgctcttcaccatgcaaatcacataatata
taagggggtgttttgtttctacggactatttttagtccctctattttattccctaaattgccaaataaggaaattaaaactctatcggtaatttagggacta
gaaactaaacaccccccccccccctaagttgaattgctggttcattgtcagagtgtttgtggttgggttttcttactttgttttgttttcgttgtattcagg
cttttatgttgaggagagcagttgaagagccactgggtcatgccgctgctatgatcaccgacgtccttgagcatgcagagtccagccatgtaatc
actgcttcgcagaagaaagccctcctccgagaaatggctctacggctgtcatgaagcaagcgccgctcattcggacgtcgcagaagaacggtt
tcctgcctctgcctgcacatcggtggttagtacgaggatctgtgatctcaacatgagctcgatcatgttgttagcagaagatacaggcccggagg
gacgtgtccacgtgcacatagagtttttttttttggggtcatgttcacgttttggaaatagcgaggcaatgttgtgtgcagctatgtatggtgtgtaga
cctcgggtaagccagttccttccttctgtgcaaaactagttacgcctgcggcctgccattccgtgttaggttgactggatggatttttagggctgcgt
cgctgccacttcggttggtgccccatatgtaaacgagatgggcttgaatgcttggttgtaacctttttttttaatgataggagctctgt ccttt-3';
SEQ ID NO. 2:
MVWLQDQLEMHKFHWYSRAPTPSEFWIPLAAWFTVGLVGLWTFFHFFSVWRRKISLSWM
KIIARSKRKNFERNHEVPTAEHVWNTESLIRAKGMMCCVCLESISPAQPLGQMMTSENMV
HRCNVCGAAAHIICSSNSQKDCKCISMFGFKHVVHQWTVLWTDIADQSEEGQYCSYCEEL
CSESFLGGPPIYCCMWCQRLVHADCQSAMATETGEICDLGPFKRLILSPLFVRAISKPGSILS
SITHGANEFATTVRGRLNRTKKEKHQNRFSSDSNDDSSSDTTLNSNQRAGELKTIGDSAQRS
PENEHDSSESDGRELISESRRLGNDETGEVKLKYALSELPADSRPLLIFINKKSGAQRGDLLK
HKLHFLLNPVQVFELSSSQGPETGLFLFRKVPHFRILVCGGDGTVGWVLDAIDKQNYESPPP
IAILPAGTGNDLSRVLSWGGGLGAVEKQGGLCTILHDIEHAAVTILDRWKVTVEDKKSKNV
LLVKYMNNYLGIGCDAKVALDIHNLREGNPEKFYSQFLNKVLYAREGAKSIIDRAFVDLP
WQVRLEVDGTEIEIPEDSEGVLVANIPSYMGGVDLWQNEGEDPEDFDPQSIHDKMLEVVSI
TGAWHLGTLQVGLSRARRIAQGQSIKIQMFAPFPVQVDGEPWVQQPCTLKISHHGQAFML
RRAVEEPLGHAAAMITDVLEHAESSHVITASQKKALLREMALRLS.

Tropical and subtropical maize germplasm contains abundant genetic variations that are lacking in temperate maize, making it a crucial germplasm resource for maize breeding. In the disclosure, 6 tropical and subtropical maize inbred lines with significant differences in kernel oil contents are used to construct a multi-parent population with rich variation in kernel oil content. From the multi-parent population consisting of the 6 parents, a functional gene, Zm00001d029550, which is closely associated with the oil content of maize kernels, is identified. The gene can explain 1.4% of the phenotypic variation in kernel oil content. Haplotype analysis shows that, in 429 RILs, the Zm00001d029550 gene has 3 haplotypes (Hap1, Hap2 and Hap3). The kernel oil content of Hap3 is significantly higher than that of Hap1 and Hap2. Therefore, Hap3 of the Zm00001d029550 gene is the haplotype that significantly increases kernel oil content. The results of the disclosure help to further study the regulatory mechanism of the oil content of maize kernels, and also provide technical support for breeding maize varieties with high quality and high oil content.

The disclosure further provides an application of the Zm00001d029550 gene described in the above technical solution in regulating oil content of plant kernels or in plant breeding. In the disclosure, the regulating the oil content of the plant kernels preferably includes: improving an expression quantity of the Zm00001d029550 gene to improve the oil content of the plant kernels, or reducing the expression quantity of the Zm00001d029550 gene to reduce the oil content of the plant kernels; the plant breeding preferably includes: cultivating plants with a high kernel oil content or cultivating plants with a low kernel oil content; the plants preferably include gramincous plants; and the gramincous plants preferably include maize.

The disclosure further provides a method for improving oil content of maize kernels, including:
over-expressing the Zm00001d029550 gene described in the above technical solution in maize.

The disclosure further provides a method for reducing oil content of maize kernels, including:
reducing the expression quantity of the Zm00001d029550 gene described in the above technical solution in maize.

In the disclosure, the method for reducing includes: silence, knock or knock down.

It is to be understandable that although increasing the oil content of maize kernels can improve the quality and product value of maize, in practical applications, the demand for maize quality traits is not uniform, so it is also possible to reduce the oil content of maize kernels to obtain maize with high starch and high protein content, which is also one of the demands.

To further illustrate the disclosure, the Zm00001d029550 gene for regulating oil content of maize kernels and an application thereof provided in the disclosure are described in detail below in combination with the accompanying drawings and embodiments, but the description is not to be construed as limiting the scope of protection of the disclosure.

### Embodiment 1

### 1.1 Plant Materials

Experimental fields were planted in three different ecological environments: Yanshan County, Yunnan Province (denoted as YS, with an altitude of 1540 m, longitude of 104.5° E, and latitude of 23.6° N) in 2021 and 2022, and Jinghong City (denoted as JH, with an altitude of 606.5 m, longitude of 100.58° E, and latitude of 21.54°N) in 2023. 5 multi-parent populations were obtained by single-grain transmission method: pop1 (Ye107×CML312), pop2 (Ye107×CML384), pop3 (Ye107×CML395), pop4 (Ye107×YML46) and pop5 (Yel07×YML32). The 6 parents (Ye107, CML312, CML384, CML395, YML46 and YML32) were disclosed in literature Jiang F, Liu L, Li Z, et al. Identification of Candidate QTLs and Genes for Ear Diameter by Multi-Parent Population in Maize. Genes (Basel). 2023; 14 (6): 1305. Published 2023 Jun. 20. doi: 10.3390/genes14061305 and Jiang F, Liu L, Li Z, et al. Identification of Candidate QTLs and Genes for Ear Diameter by Multi-Parent Population in Maize. Genes (Basel). 2023; 14 (6): 1305. Published 2023 Jun. 20. doi: 10.3390/genes 14061305. The common parent Ye107 was a key excellent inbred line developed from two lines belonging to different heterotic groups in China's breeding programs, and it had been utilized as a parent in many commercial hybrids that were cultivated across a considerable production area in China (Zhen, S., Gao, G., Wang, X., Ning, H., and Duan, X. (2004). Appraisal of drought-enduring quality of several maize inbred lines. J Maize Sci. (in Chinese) 12, 18-19.).

In 5 multi-parent populations, pop1 had 92 RILs, pop2 had 92 RILs, pop3 had 83 RILs, pop4 had 74 RILs, and pop5 had 88 RILs, and ultimately, 429 RILs with rich genetic variations were constructed. The kernel oil content of CML312 was 6.8%, the kernel oil content of CML384 was 7.03%, the kernel oil content of CML395 was 7.1%, the kernel oil content of YML46 was 5.9%, the kernel oil content of YML32 was 7.3%, and the kernel oil content of Ye107 was 3.51%. The parental information is shown in Table 1.

TABLE 1
Parental information

| | | | | Kernel |
|---|---|---|---|---|
| | | Heterotic | Ecological | oil |
| Parent | Genealogy | group | type | content |
| Ye107 | Derived from US hybrid | Reid | Temperature | 3.51% |
| | DeKalbXL80 | | | |
| CML312 | S89500-F2-2-2-1-1-B*5- | nonReid | Subtropical | 6.8% |
| | 2-1-6-1 (DH) | | | |
| CML384 | P502c1#-771-2-2-1-3- | Reid | Subtropical | 7.03% |
| | B-1-1-3-1(DH) | | | |
| CML395 | 90323B-1-B-1-B*4-1-1- | nonReid | Tropical | 7.1% |
| | 2-1(DH) | | | |
| YML46 | SW1-1-1-2-1-2-1 | Suwan | Tropical | 5.9% |
| YML32 | Suwan1(S)C9-S8-346- | Suwan | Tropical | 7.3% |
| | 2(Kei890 2)-3-4-4-6 | | | |

### 1.2 Experiment Design

A randomized complete block design (RCBD) was employed for experiments conducted in Yanshan in 2021 (denoted as 21YS), Yanshan in 2022 (denoted as 22YS), and Jinghong in 2023 (denoted as 23JH). Each location had three replicates. Each experimental field was 4.0 m in length, 0.7 m in row spacing, and 0.25 m in plant spacing, with 14 plants in each row. 10 plants were sampled from each row. The maize in the experimental fields was managed according to local standard agronomic practices. The oil content was determined using a near-infrared grain analyzer.

### 1.3 Heritability Analysis

After preliminary processing of the phenotypic data collected from the three locations at different times, the Ime4 package in R software (version 4.0.5) was utilized to perform a correlation analysis on TOC of different populations in different environments, to calculate the mean, standard deviation, skewness, kurtosis, and coefficient of variation (CV) of the oil content, followed by a normality distribution test. Reference was made to the methods in Knapp S J. Confidence intervals for heritability for two-factor mating design single environment linear models. TheorAppl Genet. 1986; 72 (5): 587-591. and Moran, P.; Smith, C. The correlation between relatives on the supposition of mendelian inheritance. Trans. Royal Soc. Edinb. 1918, 52, 438-899. to calculate broad-sense heritability, and the formula is as shown in formula I:

$${h}^{2} = \frac{σ {g}^{2}}{σ {g}^{2} + \frac{σ {ge}^{2}}{e} + \frac{{σε}^{2}}{re}} \times 100 \% , \qquad Formula I$$

where σg² represents a genetic variance, σge² represents a variance of the interaction between the environment and the genotype, σε² represents a residual, e represents environment, and r represents repetition. h² can help identify phenotypic trait variations, the larger the h² is, the stronger the genetic control of the trait is, and the smaller the influence of the environment.

### 1.4 DNA Extraction and Genomic Sequencing

First, a cetyl trimethyl ammonium bromide (CTAB) method was employed to extract genomic DNA from leaves of maize seedlings. Subsequently, genomic DNA isolated from each F9RIL was digested with restriction enzymes Pstl and MspI. Then, the DNA was ligated to a barcoded adapter (New England BioLabs) using T4 ligasc. A genotyping-by-sequencing DNA (GBSDNA) library was constructed according to a GBS protocol and was sequenced.

QIAquickPCR purification kit (QIAGEN, Valencia, California, USA) was used to combine all the linked samples and purified. Polymerase chain reaction (PCR) amplification was performed using a primer matched with the adapter. Finally, QubitdsDNAHS analysis kit (Life Technologies, Big Island, New York State, USA) was used for purifying and quantifying PCR products. After using Egel system (Life Technologies) to select 200-300 bp PCR products, Qubit 2.0 fluorimeter and Qubit dsDNAHS analysis kit (Life Technologies) were used to estimate the concentration of the library. Subsequently, TASSEL v5.0 (Li C, Guan H, Jing X, et al. Genomic insights into historical improvement of heterotic groups during modern hybrid maize breeding. Nat Plants. 2022; 8 (7): 750-763.) was used to generate sequencing reading. Before performing trait analysis by association, evolution and linkage (TASSEL) analysis, 80 poly (A) bases were added to the 3′ terminus of all sequencing readings. For comparative analysis, a B73_V4 reference genome sequence was used and analyzed using Senticon software (parameter “bwamem-k 32-M-R”) (Pei S, Liu T, Ren X, Li W, Chen C, Xic Z. Benchmarking variant callers in next-generation and third-generation sequencing analysis. Brief Bioinform. 2021; 22 (3): bbaa148). The comparative results were sorted and de-duplicated using Samtools (using parameter rmdup). Ultimately, 584,847 high-quality single nucleotide polymorphisms (SNPs) were generated, and were annotated using software ANNOVAR (Wang K, Li M, Hakonarson H. ANNOVAR: functional annotation of genetic variants from high-throughput sequencing data. Nucleic Acids Res. 2010; 38 (16): c164.).

### 1.5 Phylogenetic Tree, PCA and LD Analysis

Phylogenetic tree was analyzed using Tassel v5.0 software, and 584,847 high-quality SNPs were utilized to assess the genetic relationships among 429 RILs. PCA was conducted using R package of 4.3.2 version, and the results were visualized with the scatterplot3d package. LD decay was evaluated using the Pop LD decay v3.42 (Zhang C, Dong S S, Xu J Y, He W M, Yang T L. PopLDdecay: a fast and effective tool for linkage disequilibrium decay analysis based on variant call format files. Bioinformatics. 2019; 35 (10): 1786-1788) with the original SNP data. The parameters for calculating r² (correlation coefficient) values were set to default. The LD decay plot was generated using default parameters.

### 1.6 GWAS

GWAS was performed using an efficient mixed-model association (EMMA) analysis method in a genome-wide efficient mixed-model association (GEMMA) software package (Zhou X, Stephens M. Genome-wide efficient mixed-model analysis for association studies. Nat Genet. 2012; 44 (7): 821-824. Published 2012 Jun. 17). The analysis was performed using the following mixed-model method:

$$y = Xa + Sb + Km + e , \qquad Formula II$$

where y represents phenotype, a and b are fixed effects, representing the labeled and unlabeled effects, respectively, and m represents unknown random effect. Occurrence matrices of a, b and m are represented as X, S and K, and e represents a vector of a random residual effect. To correct population structure, in the disclosure, the first three principal components (PCs) were used to construct S matrix, and kinship (K) matrix was constructed using a simple matching coefficient matrix. The genetic relationships among individuals were modeled as random effects using the K matrix. In the association analysis, a significant threshold of P value was set as p<1×10⁻⁶ to control type I errors.

In the disclosure, PLINK software (Purcell S, Neale B, Todd-Brown K, et al. PLINK: a tool set for whole-genome association and population-based linkage analyses. Am J Hum Genet. 2007; 81 (3): 559-575) was employed to calculate independent markers, with the parameter of -indeppairwise 5050.2. Formula-log 10 (1/number of SNPs) was employed to calculate significance threshold −log 10(p) >4.5, for identifying significant SNPs associated with TOC in maize. SNPs that met or exceeded the threshold were extracted using bedtools v1.7 (Strable J, Wallace J G, Unger-Wallace E, et al. Maize YABBY Genes drooping leaf1 and drooping leaf2 Regulate Plant Architecture. Plant Cell. 2017; 29 (7): 1622-1641). On the basis of the B73 (RefGen_v4) reference genome and annotation information, candidate genes associated with TOC of maize were identified within the 20 kb upstream and downstream regions of the significantly associated SNPs. On the basis of the observation results of a plateau in the r² values at 20 kb in the LD decay plot, in the disclosure, it was decided to screen for candidate genes associated with TOC of maize within the 20 kb upstream and downstream regions of the significantly associated SNPs.

### 1.7 Construction of Genetic Maps and QTL Localization

By setting the parameter minor allele frequency (MAF) ≥0.05, the SNP data were filtered by comparing with the maize reference genome B73 (RefGen_v4). Subsequently, genetic linkage maps were constructed using JoinMap 4.0 (Ooijen, J. W., Ooijen, J. W., Verlaat, J. V., Ooijen, J. W., Tol, J., Dalen, J., Buren, J. B., Meer, J. V., Kricken, J. H., Ooijen, J. W., Kessel, J. S., Van, O., Voorrips, R. E., & Heuvel, L. P. (2006). JoinMap®4, Software for the calculation of genetic linkage maps in experimental populations) on the basis of allelic SNPs. Linkage groups were formed using a logarithm of odds (LOD) threshold ≥2.5. QTL labeling on TOC was performed using the composite interval mapping (CIM) method in Windows QTL Cartographer v2.0 (Zeng Z B. Precision mapping of quantitative trait loci. Genetics. 1994; 136 (4): 1457-1468). The LOD threshold was determined on the basis of 1000 random permutation tests, with a significance level of p≤0.05. QTLs with a LOD threshold ≥2.5 were considered significant. The proportion of phenotypic variation explained (PVE) by each QTL was measured using R². The QTL names were constructed by prefixing the letter “q” to denote a QTL, followed by an abbreviation of the trait name, the corresponding chromosome number, and the marker position (Ribaut J M, Hoisington D A, Deutsch J A, Jiang C, Gonzalez-de-Leon D. Identification of quantitative trait loci under drought conditions in tropical maize. 1. Flowering parameters and the anthesis-silking interval. Theor Appl Genct. 1996; 92 (7): 905-914).

### 1.8 Identification and Functional Annotation of Candidate Genes

The significant SNPs identified by GWAS were compared with the QTL location results to identify consistent loci. The overlapped SNPs within the QTL intervals were selected to screen for candidate genes. Candidate genes were searched within a 20 kb upstream and downstream range of the significant SNPs. The B73 v4 reference genome of maize was utilized to predict candidate genes in MaizeGDB (https://www.maizegdb.org/). Functional annotations for the candidate genes were obtained using the InterPro database.

### 1.9 Haplotype Analysis

Haplotype analysis was conducted on SNPs associated with TOC in three environments using Haploview v4.2 software. Initially, a high-density genome-wide SNP was employed to construct a haplotype map. According to the positions of these loci and the results of LD analysis, haplotypes of SNPs significantly associated with maize oil content were determined. Finally, genes within the haplotypes were annotated, to determine functionally related genetic loci.

### 2. Results

### 2.1 Phenotypic Analysis of Kernel Oil Content

In three different ecological environments (22YS, 23JH and 21YS), phenotypic investigations for TOC in maize were performed using five RIL populations, and corresponding data were collected. Table 2 presents the descriptive statistical results of the phenotypic data for the five RIL populations that show significant effects on TOC. The CV values for pop1 in three environments were 12.6%, 12% and 13.7%, respectively; the CV values for pop2 in three environments were 11.4%, 10.9% and 12.4%, respectively; the CV values for pop3 in three environments were 14.7%, 11.7% and 12.1%, respectively; the CV values for pop4 in three environments were 8.8%, 7.6% and 8.9%, respectively; and the CV values for pop5 in three environments were 11.2%, 10.4% and 9.8%, respectively. The absolute values of skewness and kurtosis for all five RIL populations were less than 1, indicating a low degree of bias. The phenotypic frequency distributions of pop1, pop2, pop3, pop4 and pop5 approximated a normal distribution. Under the three environmental conditions, the broad-sense heritability of the RIL populations of pop2 and pop5 was higher, with values of 93.1% and 91.6%, respectively. The genotype-by-environment interaction differences were statistically significant.

TABLE 2
Phenotypic analysis results of oil content

| | | Mean | Standard | | | | Broad-sense | |
|---|---|---|---|---|---|---|---|---|
| Population | Environment | value | deviation | Skewness | Kurtosis | CV | heritability | Correlation |
| pop1 | 22YS | 5.273 | 0.662 | −0.158 | −0.481 | 12.60% | 88.1 | 22YS/23JH = 0.77** |
| | 23JH | 5.189 | 0.623 | 0.375 | −0.226 | 12% | | 23JH/21YS = 0.73** |
| | 21YS | 5.199 | 0.711 | 0.07 | −0.08 | 13.70% | | 21YS/22YS = 0.68** |
| pop2 | 22YS | 5.164 | 0.59 | 0.146 | −0.01 | 11.40% | 93.1 | 22YS/23JH = 0.89** |
| | 23JH | 5.05 | 0.55 | 0.314 | −0.334 | 10.90% | | 23JH/21YS = 0.91** |
| | 21YS | 4.91 | 0.61 | 0.203 | −0.217 | 12.40% | | 21YS/22YS = 0.72** |
| pop3 | 22YS | 5.501 | 0.809 | −0.243 | −0.071 | 14.70% | 83.9 | 22YS/23JH = 0.87** |
| | 23JH | 5.32 | 0.623 | 0.052 | −0.441 | 11.70% | | 23JH/21YS = 0.79** |
| | 21YS | 5.12 | 0.62 | 0.019 | −0.387 | 12.10% | | 21YS/22YS = 0.47** |
| pop4 | 22YS | 4.884 | 0.432 | 0.533 | −0.045 | 8.80% | 79.4 | 22YS/23JH = 0.78** |
| | 23JH | 4.888 | 0.371 | 0.163 | −0.925 | 7.60% | | 23JH/21YS = 0.76** |
| | 21YS | 4.912 | 0.439 | 0.353 | −0.087 | 8.90% | | 21YS/22YS = 0.40** |
| pop5 | 22YS | 5.276 | 0.592 | 0.241 | −0.402 | 11.20% | 91.6 | 22YS/23JH = 0.91** |
| | 23JH | 5.214 | 0.542 | 0.242 | −0.644 | 10.40% | | 23JH/21YS = 0.88** |
| | 21YS | 5.167 | 0.504 | 0.222 | −0.274 | 9.80% | | 21YS/22YS = 0.70** |

Note:
22YS represents the experiment conducted in Yanshan in 2022, 23JH represents the experiment conducted in Jinghong in 2023, and 21YS represents the experiment conducted in Yanshan in 2021; and
**indicates P < 0.01.

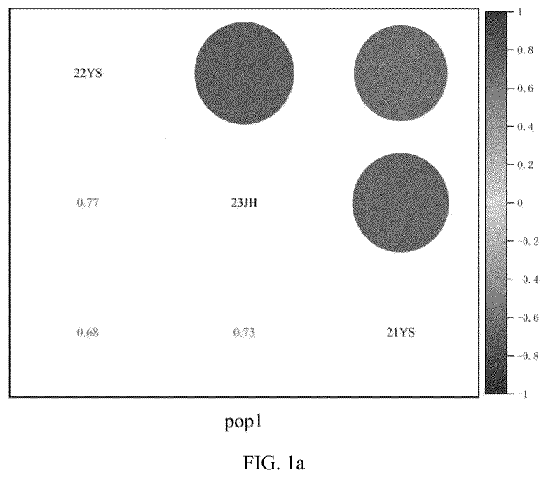

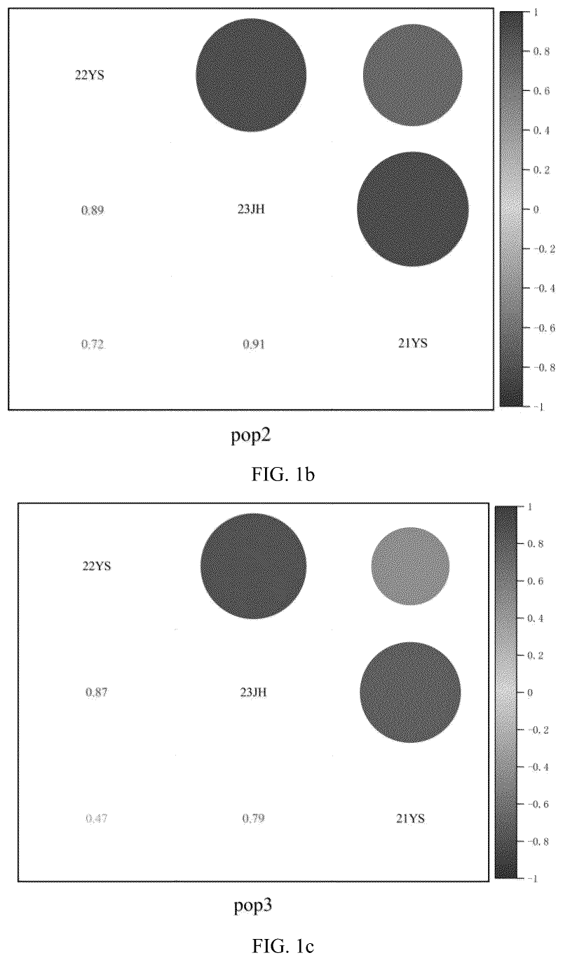

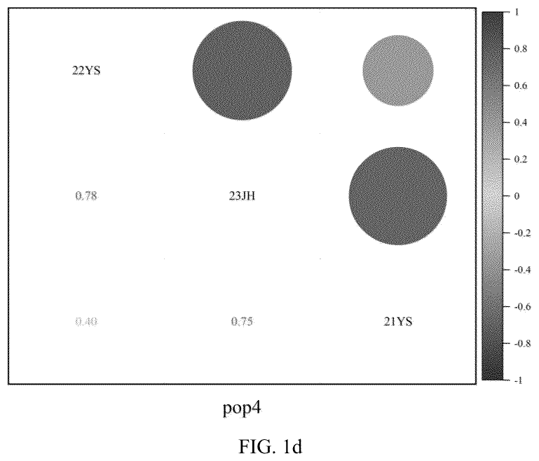

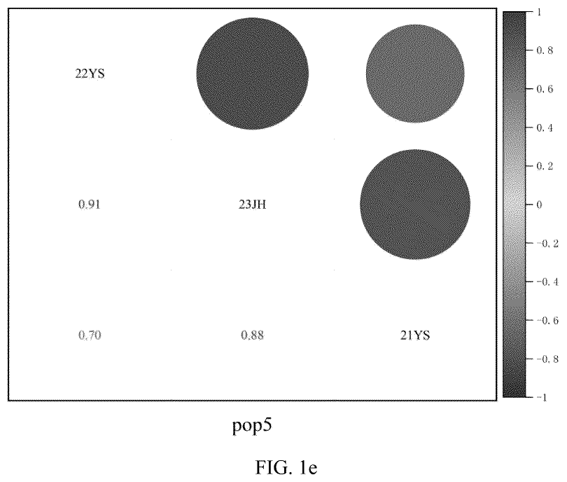

FIG. 1 shows the correlation analysis of TOC in different environments within the same population. In the 22YS, 23JH and 21YS environments, the correlation coefficients for TOC in pop1 were 0.77, 0.73 and 0.68, respectively; the correlation coefficients for TOC in pop2 were 0.89, 0.91 and 0.72, respectively; the correlation coefficients for TOC in pop3 were 0.87, 0.79 and 0.47, respectively; the correlation coefficients for TOC in pop4 were 0.78, 0.76 and 0.40, respectively; and the correlation coefficients for TOC in pop5 were 0.91, 0.88 and 0.70, respectively. The consistently high correlation coefficients in the three environments suggested that the responses of the five RIL populations to TOC were significant and stable, ensuring the phenotypic reliability for subsequent GWAS analysis.

### 2.2 QTL Location in RIL Populations

Pop1 had a total of 981 high-quality SNPs, with an average distance of 1.07 cM between SNPs, and a total genetic distance of 1045.83 cM in chromosomes. Pop2 had a total of 693 high-quality SNPs, with an average distance of 0.83 cM between SNPs, and a total genetic distance of 575.78 cM in chromosomes. Pop3 had a total of 2021 high-quality SNPs, with an average distance of 2.27 cM between SNPs, and a total genetic distance of 4953.45 cM in chromosomes. Pop4 had a total of 857 high-quality SNPs, with an average distance of 0.94 cM between SNPs, and a total genetic distance of 802.12 cM in chromosomes. Pop5 had a total of 638 high-quality SNPs, with an average distance of 0.91 cM between SNPs, and a total genetic distance of 581.28 cM in chromosomes. QTL location and effect analysis for TOC were conducted on pop1, pop2, pop3, pop4 and pop5 in three different environments. SNP markers with a deletion rate of 10% or more and loci with an MAF less than 5% were filtered out. The LOD threshold was set to ≥2.5.

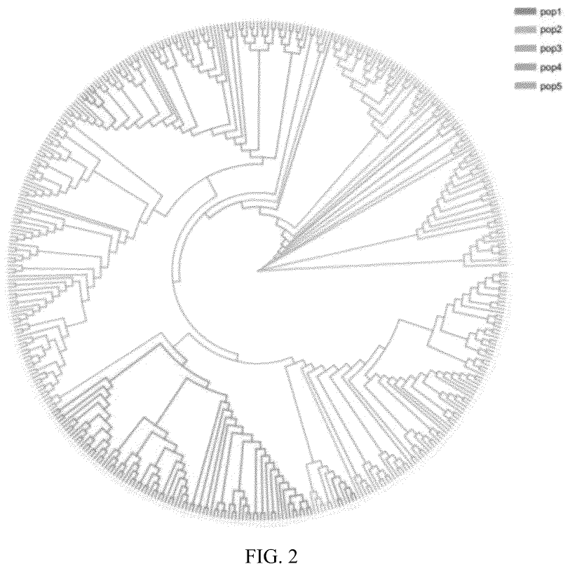

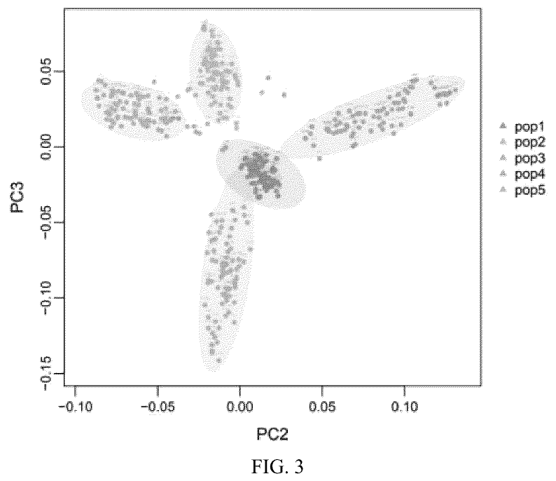

For pop1, a total of 5 TOC QTLs, namely, qTOC2-1, qTOC2-2, qTOC2-3, qTOC3-1 and qTOC9-1, were detected in three different environments, explaining 16.3%, 13%, 13%, −9.8% and −10.6% of the phenotypic variation, respectively (FIGS. 2-4, and Table 3). Among them, the qTOC2-2 identified on chromosome 2 had the highest LOD value of 4.63 and an additive effect value of 0.06. The additive effect values of qTOC2-1, qTOC2-3, qTOC3-1 and qTOC9-1 were all positive. Pop2 had no significant QTLs. For pop3, a total of 5 TOC QTLs, namely, qTOC1-1, qTOC1-2, qTOC4-1, qTOC4-2 and qTOC7-1, were detected in three different environments, explaining 13.1%, 1.4%, 19.9%, 14.3% and 15.1% of the phenotypic variation, respectively. Among them, the qTOC4-1 identified on chromosome 4 had the highest LOD value of 5.65 and the largest additive effect value of 0.65. The additive effect values of qTOC1-1, qTOC1-2 and qTOC4-2 were all positive. For pop4, a total of 3 TOC QTLs, namely, qTOC5-1, qTOC7-1 and qTOC8-1, were detected in three different environments, explaining 3.8%, −14.6% and −16.6% of the phenotypic variation, respectively. Among them, the qTOC8-1 identified on chromosome 8 had the highest LOD value of 4.61 and the largest additive effect value of 0.124. The additive effect values of qTOC5-1 and qTOC7-1 were both positive. For pop5, a total of 5 TOC QTLs, namely, qTOC2-1, qTOC2-2, qTOC3-1, qTOC5-1 and qTOC5-2, were detected in three different environments, explaining 14.4%, 23.1%, 17.7%, 14.6% and 10.3% of the phenotypic variation, respectively. Among them, the qTOC2-2 identified on chromosome 2 had the highest LOD value of 5.73 and the largest additive effect value of 0.36. The additive effect values of qTOC2-2 and qTOC5-1 were positive.

TABLE 3
Positions and effects of QTL of oil content detected in RIL populations

| | | | | | | | Phenotypic |
|---|---|---|---|---|---|---|---|
| | | | Physical | Mapping | LOD | Additive | interpretation |
| Population | QTL | Chromosome | position | interval | threshold | effect | rate |
| pop1 | qTOC2-1 | 2 | 115 | 29801036- | 2.9 | 0.09 | 0.163 |
| | | | | 37206712 | | | |
| | qTOC2-2 | 2 | 106.98 | 14177571- | 4.63 | 0.06 | 0.13 |
| | | | | 47384437 | | | |
| | qTOC2-3 | 2 | 117.03 | 14177571- | 3.75 | 0.05 | 0.13 |
| | | | | 37206712 | | | |
| | qTOC3-1 | 3 | 33.25 | 193353260- | 3.82 | 0.03 | −0.098 |
| | | | | 227027669 | | | |
| | qTOC9-1 | 9 | 75.92 | 110367967- | 3.39 | 0.03 | −0.106 |
| | | | | 125469868 | | | |
| pop3 | qTOC1-1 | 1 | 27.11 | 273307800- | 3.81 | 0.4 | 0.131 |
| | | | | 273997185 | | | |
| | qTOC1-2 | 1 | 198.72 | 61742795- | 3.82 | 0.25 | 0.014 |
| | | | | 76153784 | | | |
| | qTOC4-1 | 4 | 232.73 | 161621304- | 5.65 | 0.65 | 0.199 |
| | | | | 164227248 | | | |
| | qTOC4-2 | 4 | 599.88 | 172418126- | 3.46 | 0.32 | 0.143 |
| | | | | 175502163 | | | |
| | qTOC7-1 | 7 | 199.1 | 50416681- | 4.33 | −0.56 | 0.151 |
| | | | | 50735457 | | | |
| pop4 | qTOC5-1 | 5 | 34.22 | 79346283- | 3.31 | 0.01 | 0.038 |
| | | | | 81012543 | | | |
| | qTOC7-1 | 7 | 29.17 | 132794775- | 3.01 | 0.11 | −0.146 |
| | | | | 153544218 | | | |
| | qTOC8-1 | 8 | 89.73 | 10663910- | 4.61 | 0.124 | −0.166 |
| | | | | 30775820 | | | |
| pop5 | qTOC2-1 | 2 | 1.57 | 222088040- | 3.75 | −0.29 | 0.144 |
| | | | | 232416169 | | | |
| | qTOC2-2 | 2 | 15.67 | 159679997- | 5.73 | 0.36 | 0.231 |
| | | | | 163407700 | | | |
| | qTOC3-1 | 3 | 33.53 | 114248818- | 4.27 | −0.24 | 0.177 |
| | | | | 166417378 | | | |
| | qTOC5-1 | 5 | 22.99 | 135409128- | 3.92 | 0.28 | 0.146 |
| | | | | 153772976 | | | |
| | qTOC5-2 | 5 | 55.99 | 31369431- | 2.8 | −0.23 | 0.103 |
| | | | | 38866833 | | | |

### 2.3 Population Structure of RIL Population

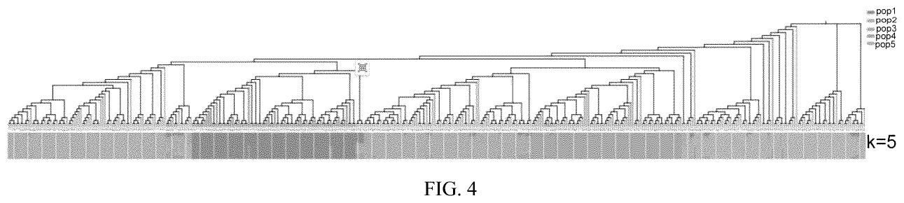

In the disclosure, Admixture software was used for analyzing population structures of 429 materials, with the analysis results shown in FIGS. 2-4. Overall, the results of population structure, PCA, and genetic distance or correlation (FIGS. 2-4) were consistent. In the PCA plot, scattered points could originate from heterogeneity within the population or outliers. On the basis of genealogy or genetic background, RILs could be classified into five major clusters. When K=5 (FIG. 4), the population structure of the RILs became clear, with 92 RILs in pop1, 92 RILs in pop2, 83 RILs in pop3, 74 RILs in pop4, and 88 RILs in pop5. Phylogenetic tree analysis also revealed five genetic clusters, which were consistent with the population structure based on kinship.

2.4 LD decay

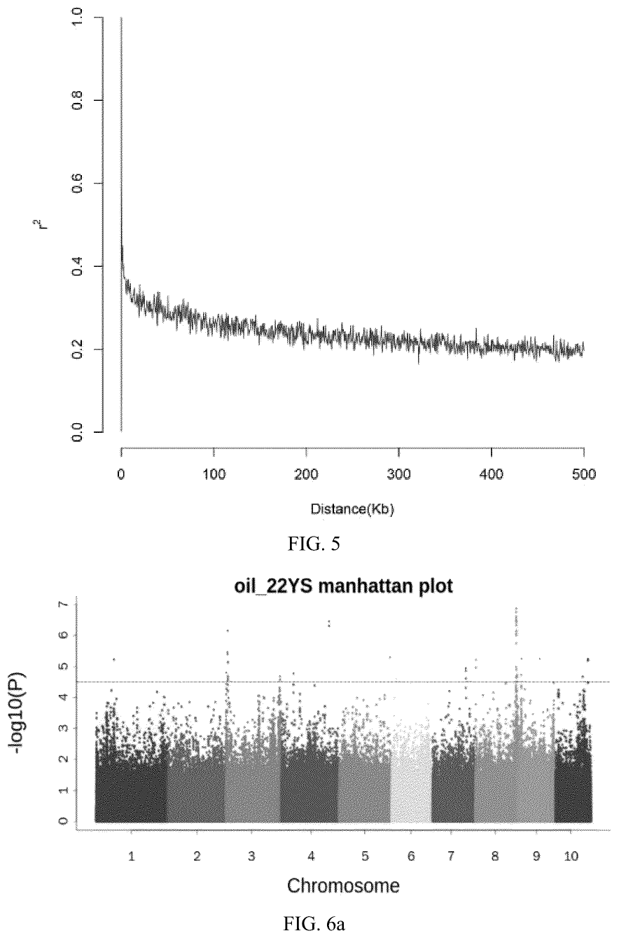

In the disclosure, the original SNP dataset was utilized to conduct LD decay analysis. The LD decay for each population was calculated, and it was found that when the decline rate of r² tended to stable, the physical distance was approximately 20 kb (FIG. 5). The slow decay of LD suggested that higher levels of domestication and stronger selection intensity had led to reduced genetic diversity.

### 2.5 GWAS of TOC

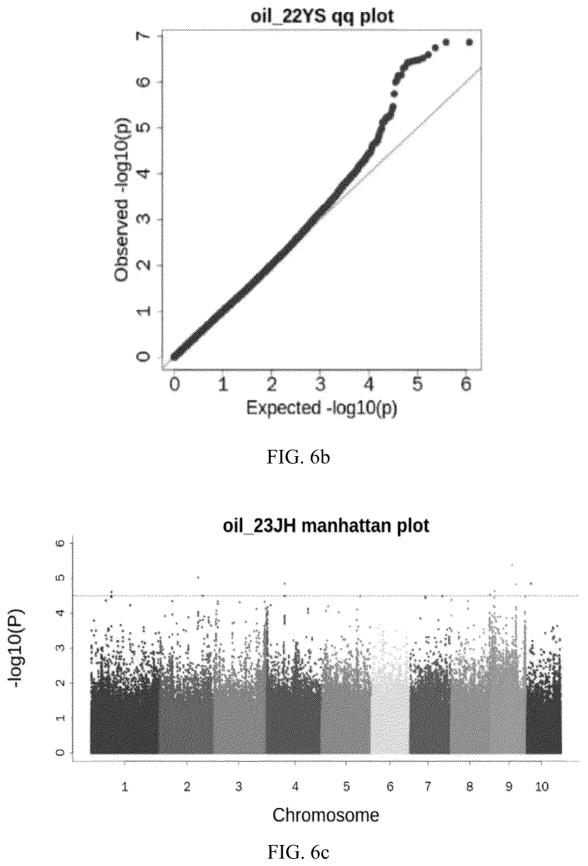

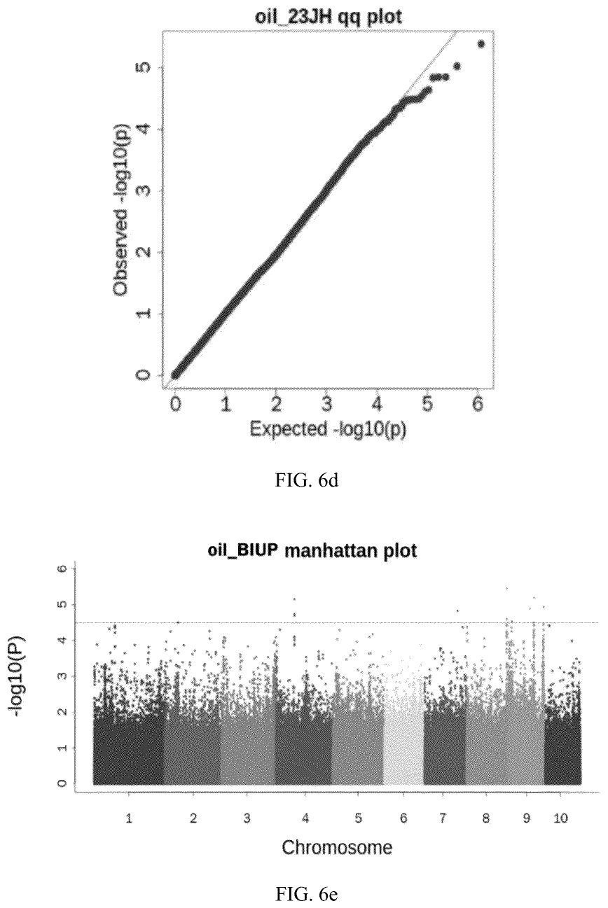

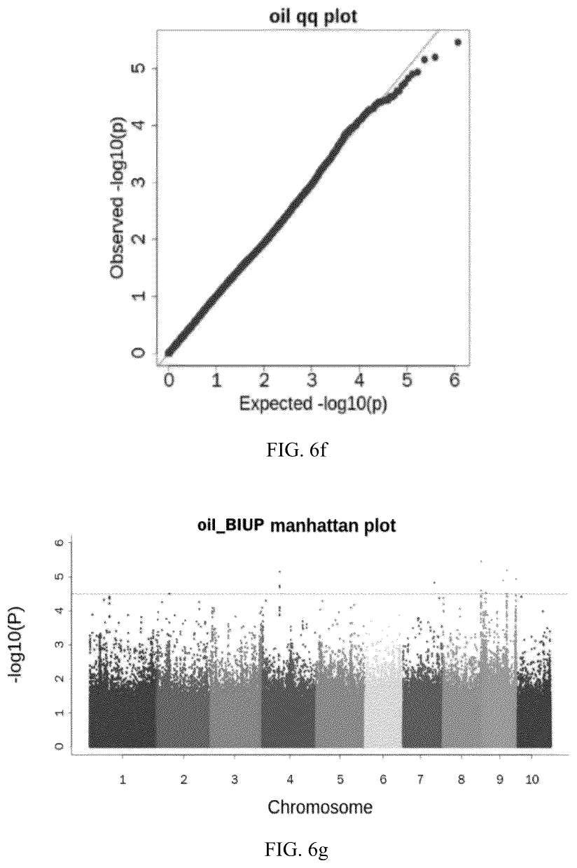

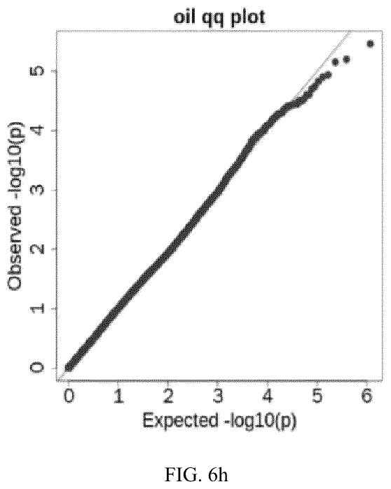

In the disclosure, GWAS analysis was conducted using 429 samples and 584,847 valid SNP markers (with MAF ≥5% and deletion values r²<0.8). A threshold of −log 10(P) >4.5 was set, and a total of 51 SNPs significantly associated with TOC were identified (FIG. 6a-FIG. 6h). Among them, 23 SNPs were detected in the 22YS environment, with the maximum additive effect value being 0.35 and the maximum dominant effect value being 0.35. In the 23JH environment, 8 SNPs were identified, with the maximum additive effect value being 0.26 and the maximum dominant effect value being 0.21. In the 21YS environment, 20 SNPs were identified, with the maximum additive effect value being 0.29 and the maximum dominant effect value being 0.34. These SNP loci were located on chromosomes 1, 2, 3, 4, 5, 6, 7, 8, 9 and 10 (Table 4). Three SNP loci on chromosomes 4, 9 and 10 were detected in all three environments. The PVE by these significant SNPs ranged from 2.8% to 13.2%, with an average value of 6.6%.

TABLE 4
SNP details significantly associated with TOC traits

| | | | | | | | | Phenotypic |
|---|---|---|---|---|---|---|---|---|
| | | | | | | Additive | dominant | interpretation |
| Environment | Chromosome | SNP | ref | alt | −log(P) | effect | effect | rate |
| 22YS | 1 | 75791466 | G | T | 5.23 | −0.26 | 0.35 | 0.035 |
| 22YS | 3 | 2108126 | G | A | 4.8 | 0.2 | 0.2 | 0.075 |
| 22YS | 3 | 7453745 | G | A | 5.46 | 0.3 | 0.04 | 0.053 |
| 22YS | 3 | 8646933 | C | A | 6.15 | 0.33 | −0.11 | 0.046 |
| 22YS | 3 | 9226566 | G | T | 4.54 | −0.28 | −0.21 | 0.036 |
| 22YS | 3 | 9371935 | C | T | 5.14 | −0.29 | −0.2 | 0.038 |
| 22YS | 3 | 230340051 | C | T | 4.68 | −0.24 | 0.14 | 0.062 |
| 22YS | 3 | 230499437 | G | A | 4.57 | 0.25 | 0.04 | 0.028 |
| 22YS | 4 | 52876804 | A | G | 4.77 | −0.19 | −0.48 | 0.035 |
| 22YS | 4 | 203717068 | A | G | 6.45 | −0.41 | −0.01 | 0.073 |
| 22YS | 5 | 216419106 | A | T | 5.3 | −0.26 | −0.05 | 0.049 |
| 22YS | 6 | 19088018 | C | T | 4.63 | NaN | NaN | 0.029 |
| 22YS | 7 | 140826856 | C | T | 4.94 | 0.31 | 0.06 | 0.042 |
| 22YS | 8 | 1952449 | C | T | 5.22 | 0.15 | −0.13 | 0.038 |
| 22YS | 8 | 172972407 | T | C | 4.99 | −0.27 | 0.13 | 0.055 |
| 22YS | 8 | 173247098 | A | T | 6.86 | −0.37 | −0.18 | 0.07 |
| 22YS | 8 | 174055891 | T | C | 6.32 | −0.27 | −0.48 | 0.098 |
| 22YS | 8 | 177414430 | C | T | 4.71 | −0.22 | −0.01 | 0.077 |
| 22YS | 9 | 13835261 | C | T | 4.72 | −0.25 | 0 | 0.092 |
| 22YS | 9 | 14820336 | C | A | 5.25 | 0.24 | −0.14 | 0.055 |
| 22YS | 9 | 92493718 | T | C | 5.24 | −0.57 | −0.16 | 0.053 |
| 22YS | 10 | 115482753 | G | A | 4.67 | 0.23 | 0.27 | 0.11 |
| 22YS | 10 | 138012512 | T | G | 5.23 | 0.35 | −0.16 | 0.117 |
| 23JH | 1 | 89810991 | G | T | 4.61 | −0.36 | −0.11 | 0.053 |
| 23JH | 2 | 172364269 | T | C | 5.02 | NaN | NaN | 0.083 |
| 23JH | 4 | 80064051 | C | A | 4.84 | 0.26 | 0.12 | 0.034 |
| 23JH | 8 | 174055891 | T | C | 4.54 | −0.2 | −0.43 | 0.081 |
| 23JH | 9 | 14820336 | C | A | 4.64 | 0.23 | −0.2 | 0.052 |
| 23JH | 9 | 92493718 | T | C | 5.38 | −0.8 | −0.47 | 0.066 |
| 23JH | 9 | 110672521 | T | C | 4.83 | −0.25 | 0.21 | 0.071 |
| 23JH | 10 | 17500491 | C | G | 4.85 | NaN | NaN | 0.057 |
| 21YS | 2 | 5102776 | G | T | 5.08 | −0.27 | −0.58 | 0.064 |
| 21YS | 2 | 62659851 | G | A | 5.21 | NaN | NaN | 0.088 |
| 21YS | 4 | 131018543 | C | A | 5.4 | NaN | NaN | 0.072 |
| 21YS | 4 | 132312280 | C | T | 4.76 | NaN | NaN | 0.055 |
| 21YS | 5 | 221373665 | G | A | 6.13 | 0.22 | −0.01 | 0.059 |
| 21YS | 5 | 222162464 | T | C | 4.7 | 0.17 | −0.01 | 0.044 |
| 21YS | 7 | 21816794 | C | G | 5 | 0.24 | −0.13 | 0.073 |
| 21YS | 7 | 165218537 | A | G | 5.96 | 0.26 | −0.17 | 0.084 |
| 21YS | 9 | 40627693 | G | A | 5.45 | NaN | NaN | 0.075 |
| 21YS | 9 | 54914157 | T | C | 4.64 | NaN | NaN | 0.057 |
| 21YS | 9 | 108901209 | A | G | 4.89 | 0.2 | 0.34 | 0.053 |
| 21YS | 9 | 108933426 | A | G | 7.44 | 0.29 | −0.09 | 0.108 |
| 21YS | 9 | 109017561 | A | G | 5.5 | 0.21 | −0.15 | 0.075 |
| 21YS | 9 | 109122650 | G | A | 4.58 | −0.2 | 0.14 | 0.08 |
| 21YS | 9 | 109283271 | G | A | 4.94 | −0.16 | −0.34 | 0.086 |
| 21YS | 9 | 109407646 | C | T | 5.07 | 0.22 | 0.16 | 0.063 |
| 21YS | 9 | 109451171 | G | C | 4.94 | −0.2 | 0.09 | 0.057 |
| 21YS | 9 | 110611574 | G | T | 5.7 | 0.29 | −0.32 | 0.132 |
| 21YS | 9 | 110672521 | T | C | 5.71 | −0.25 | −0.19 | 0.11 |
| 21YS | 10 | 17500491 | C | G | 4.6 | NaN | NaN | 0.074 |

### 2.6 Identification of Candidate Genes Related to TOC

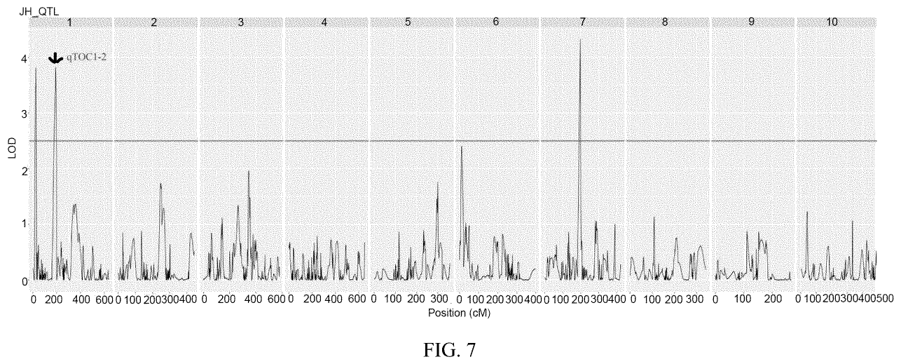

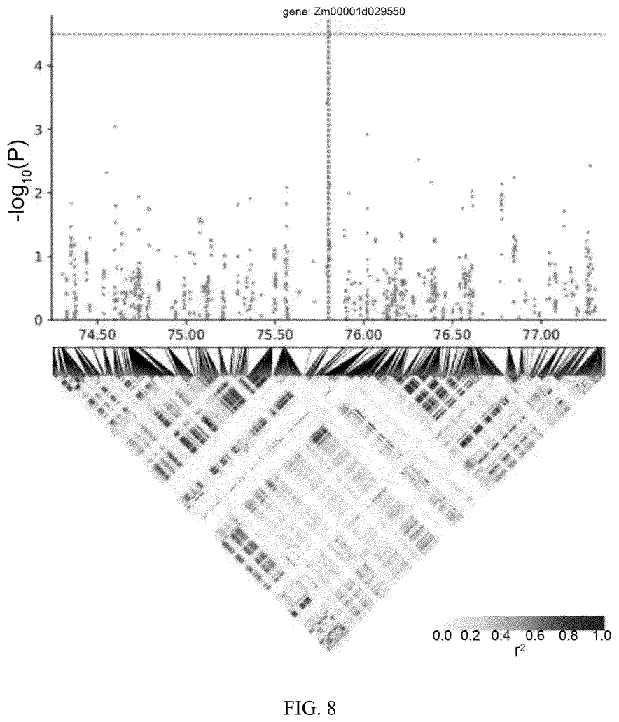

One significant QTL: qTOC1-2, was detected on chromosome 1 of pop3 in the JH environment (FIG. 7), which explained 1.4% of the phenotypic variation in TOC. The SNP_75791466 identified by GWAS overlapped with the QTL interval of qTOC1-2 (Table 5, and FIG. 8) on chromosome 1. The SNP was highly significant, as evidenced by a −log 10(p) value of 5.24. SNP_75791466 was located on 5648 bp downstream of the gene Zm00001d029550, which was considered as a second messenger for diacylglycerol (DAG) that served as an activator of protein kinase C.

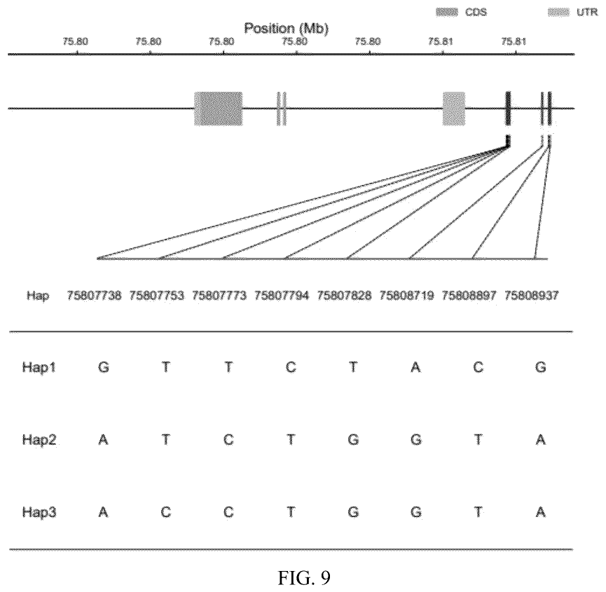

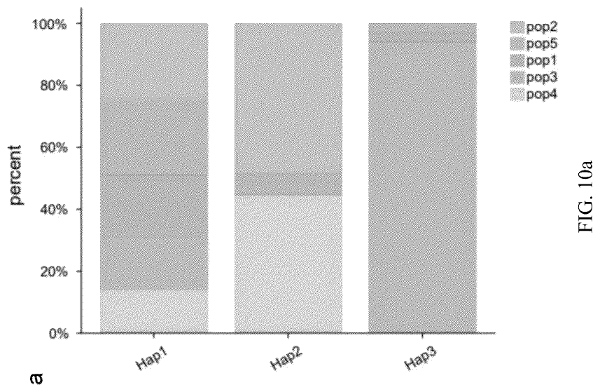

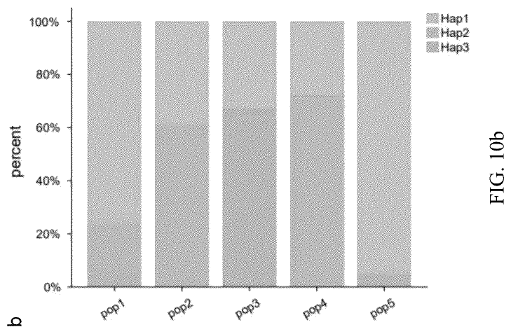

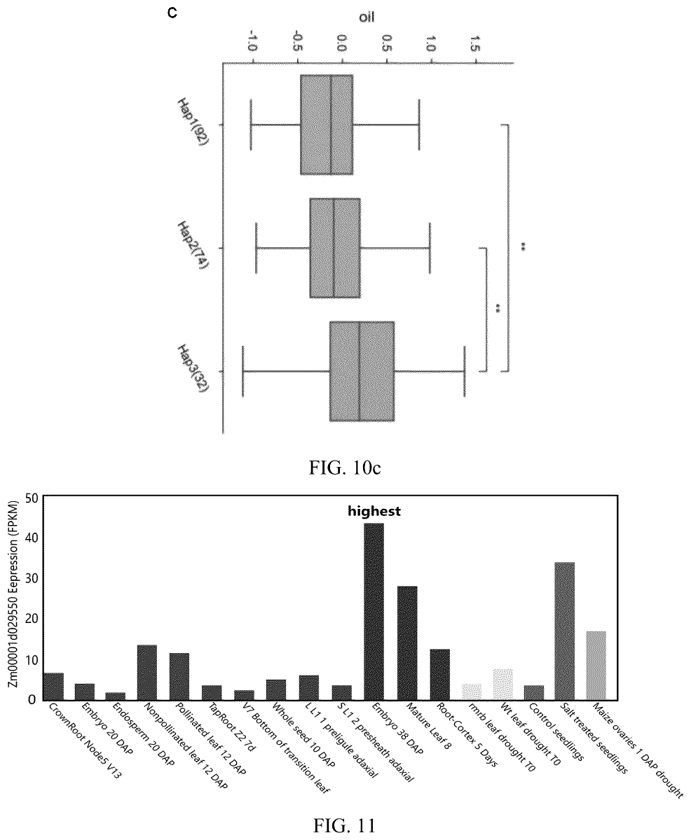

Haplotype analysis results shown that the Zm00001d029550 gene exhibited three haplotypes: Hap1, Hap2 and Hap3 (FIG. 9 and FIG. 10c). The frequency distributions of these haplotypes were as follows: Hap1 (92 of 429 RILs), Hap2 (74), and Hap3 (32). There were significant differences between Hap1 and Hap3 (p<0.001), as well as between Hap2 and Hap3 (p<0.01). The TOC of Hap3 was significantly higher than that of Hap1 and Hap2, with Hap1 having the lowest TOC. Therefore, Hap3 was regarded as the superior haplotype for increasing TOC in maize by Zm00001d029550 gene. As shown in FIGS. 10a and 10b, Hap1 haplotype was present in pop1, pop2, pop3, pop4 and pop5, the Hap2 haplotype was present in pop1, pop2 and pop4, and the Hap3 haplotype was present in pop1, pop3 and pop5. The Zm00001d029550 gene exhibited lower expression levels in various tissues, with the highest expression level observed in the embryo after maize pollination (FIG. 11), indicating that the embryo was the primary site for oil accumulation and was involved in oil synthesis and metabolism.

TABLE 5
Co-localization of gene

| SNP | | Physical | Peak | Mapping | Candidate | | | Gene | Additive | Dominant |
|---|---|---|---|---|---|---|---|---|---|---|
| marker | Chromosome | location | value | interval | gene | Start | End | annotation | effect | effect |
| SNP- | 1 | 75791466 | 5.24 | [75771466- | Zm00001d029550 | 75797114 | 75806603 | DAG | −0.271 | 0.4391 |
| 75791466 | | | | 75811466] | | | | Kinase 1 | | |
| | | | | | Zm00001d029551 | 75803417 | 75804837 | NaN | | |

The nucleotide sequence of the candidate gene Zm00001d029551 is as shown in SEQ ID NO.3. The specific sequence is as follows:

SEQ ID NO. 3:
5′-atgcaccagcacctccgtcgctccaacgccctgagcgcctccgtcgtttgattccggtttgcttctggtttctgccgcacaag
gtacgtttcattctggtttctgccgcacaagatccgctacctcttggacccggacatctacaccgacggcaggatctccgtgcagggcatcgacgt
cgtgctcttcgcgccggacgacgccaaggccacgcagaccgccgacccgaacaggaagaagatcaagctccatgcagggcatcacccaag
gccacgcagaccgccggacgactccaaggccggatctccgtgcagccgtgcagggcatcacccacaagaagaagatcaagctccggcgag
gtcagtacctacctaccttcagatctggacctgctcctttgtgtcctagtcgtaccattacagattgctctgtacatcgtagattggcagtgttctgaata
tctatagcaggcctgatacttcctatatttgtttcattgctacatacatattaattacacgaacattcgatctataaaaaaagtacaagtgatactgtaata
tacgtgaggaagtatcggaggatactgcaatctgaaaaaaattgaaatatgatactaaaatatgagatttgcaattcatgtagattgatttgcaatctg
aaaaaagattggggaggtgtgtagattaatttcacaactgatcgctagcaccaatcaatgccagttcatctagtggtcagaatgtattggatttggag
gaaggcatatgggcttgtcggtgtccgtctggacatttcctattgacatggacattttcttcttcctgtgaacgcatatctccatatttttcttctatgttttt
cattattcctagagctagttgccagtggctatattttggagataaggtagtcaaagtatgttaagttaggtgtcataagcttaacatccacttgtaggttc
caatgattggaattttttgcataccgacttctgattgatttgttgcatgtttgttttaacactatgcttccttgcagtacctctttccatctcctgtcgctgcct
ctaccatatctatctaaaggaacaaatttgagcgcatcttctgttgacagtgactttagtttgctattggtgctccagtaagaaaaatagatgtattttgtt
gatggtggcttcacaatctggcacatgactcatgaaaagctttgtttgacaaactattgtgaaggaacaagtatactgtcttttactacttcagtttgctt
gacaaactattgtgatgatggataaagtgtgatgtgatgatggataaaccaatttactatgcttcagtttactttgtttgatttttcctgattttttgaagtatt
atttatagcatcacatagctatggatgaaccattttctgatgat-3'.

Although the disclosure has been described via the above embodiment in detail, the embodiment is merely some, rather than all embodiments of the disclosure. A person can obtain other embodiments according to the embodiment without creative efforts, and these embodiments all belong to the scope of protection of the disclosure.

1. A method for identifying maize kernel oil content, the method comprising:
extracting genomic DNA of maize,
sequencing the genomic DNA of maize, and
analyzing a haplotype of Zm00001d029550 gene,
wherein the nucleotide sequence of the Zm00001d029550 gene is set forth in SEQ ID NO.1; and
wherein the haplotype of Zm00001d029550 gene comprises Hap1 of SEQ ID NO: 4, Hap2 of SEQ ID NO: 5 and Hap3 of SEQ ID NO: 6, with kernel oil content of the Hap3 being higher than that of the Hap1 and the Hap2.
